(19) United States
(12) Patent Application Publication
(10) Pub. No.: US 2017/0125067 A1
(43) Pub. Date: May 4, 2017

(54) GAS LEAK DETECTION IN DATA STORAGE DEVICE

(71) Applicant: HGST Netherlands B.V., Amsterdam (NL)

(72) Inventors: Yuka Morimoto, Kanagawa-ken (JP); Yuichi Aoki, Tokyo (JP); Kenji Kuroki, Kanagawa-ken (JP); Kohtaroh Nakano, Kanagawa-ken (JP)

(21) Appl. No.: 14/924,903

(22) Filed: Oct. 28, 2015

(51) Int. Cl. G11B 33/14; G01N 33/00; G11B 5/60

(52) U.S. Cl. CPC G11B 33/1486; G11B 5/607; G01N 33/0036

(57) ABSTRACT

An example storage device may include a sealed enclosure that encloses an atmosphere comprising a gas. The sealed enclosure may enclose a storage medium and a read-write head including a thermal flying-height control (TFC) heater and an embedded contact sensor (ECS) including a DC resistance (DCR) sensor. A DC resistance exhibited by the DCR sensor may be indicative of a temperature sensed adjacent the DCR sensor. The storage device may include a controller. The controller may cause a current to be applied to the TFC heater to generate heat, receive a signal from the ECS indicative of the temperature sensed by the DCR sensor, and determine a composition of the atmosphere or a concentration of the gas based on the signal.

### TECHNICAL FIELD

This disclosure relates to detecting gas leakage in data storage devices.

### BACKGROUND

Storage devices may include storage media such as magnetic disks. Storage devices may be unsealed and open to the surrounding atmosphere, or may be sealed from the surrounding atmosphere. Sealed storage devices may enclose an atmosphere including known concentrations of predetermined gases, such as air, nitrogen, helium, or the like. Leakage of the surrounding atmosphere into sealed storage devices may cause performance degradation and may eventually lead to failure.

### SUMMARY

In one example, the disclosure describes a storage device including a sealed enclosure enclosing a storage medium and a read-write head in an atmosphere comprising a gas. The read-write head may include a thermal flying-height control (TFC) heater and an embedded contact sensor (ECS) including a DC resistance (DCR) sensor. A DC resistance exhibited by the DCR sensor may be indicative of a temperature sensed adjacent the DCR sensor. The storage device may include a controller configured to cause a current to be applied to the TFC heater to generate heat, receive a signal from the ECS indicative of the temperature sensed by the DCR sensor, and determine a composition of the atmosphere or a concentration of the gas based on the signal.

In another example, the disclosure describes a technique including causing, by a controller, a current to be applied to a thermal flying-height control (TFC) heater. A sealed enclosure may enclose in an atmosphere comprising a gas a storage medium and a read-write head, the read-write head including the TFC heater and an embedded contact sensor (ECS) including a DC resistance (DCR) sensor. The technique also may include receiving, by the controller, from the ECS, a signal indicative of a DC resistance of the DCR sensor, wherein the DC resistance is indicative of a temperature adjacent the DCR sensor. The technique further may include determining, by the controller, a composition of the atmosphere or a concentration of the gas based on the signal.

In another example, the disclosure describes a system including a storage device including a sealed enclosure enclosing a storage medium and a read-write head in an atmosphere comprising a gas. The read-write head may include a means for electrically heating a region adjacent the read-write head and a means for exhibiting a DC resistance (DCR) indicative of a temperature sensed adjacent the read-write head. The system also may include a controller including a means for applying a current to be applied to the electrical means, a means for receiving a signal from the means for exhibiting the DC resistance, and a means for determining a composition of the atmosphere or a concentration of the gas based on the signal.

In another example, the disclosure describes a computer readable storage medium including instructions that, when executed, cause at least one processor to cause a current to be applied to a thermal flying-height control (TFC) heater. A sealed enclosure may enclose a storage medium and a read-write head in an atmosphere comprising a gas. The read-write head may include the TFC heater and an embedded contact sensor (ECS) including a DC resistance (DCR) sensor. The instructions, when executed, also may cause the at least one processor to receive, from the ECS, a signal indicative of a DC resistance of the DCR sensor. The DC resistance may be indicative of a temperature adjacent the DCR sensor. The instructions, when executed, additionally may cause the at least one processor to determine a composition of the atmosphere or a concentration of the gas based on the signal.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual and schematic block diagram illustrating an example storage device enclosing a storage medium and a read-write head.

FIG. 1B is a conceptual and schematic block diagram illustrating a cross-sectional view of the read-write head of FIG. 1A.

FIG. 2 is a flow diagram illustrating an example technique for detecting a composition of an atmosphere or a concentration of a gas within a sealed enclosure of a storage device.

FIGS. 3A-3C are charts presenting an example DC resistance versus energy supplied to a thermal flying height control heater as a function of helium concentration, and at different positions of the read-write head with respect to the disk and the ramp.

FIG. 4 is a chart presenting an example change in DC resistance versus energy supplied to a thermal flying height control heater as a function of of TFC power for different helium concentrations.

FIG. 5 is a chart presenting an example percent change in ΔDC resistance divided by change in energy supplied to a thermal flying height control heater for different helium concentrations.

FIG. 6 is a chart presenting an example change in ΔDC resistance divided by change in energy supplied to a thermal flying height control heater versus temperature for different helium concentrations.

FIG. 7A is a chart presenting an example change in ΔDC resistance divided by change in energy supplied to a thermal flying height control heater versus helium concentration during calibration.

FIG. 7B is a chart presenting an example change in ΔDC resistance divided by change in energy supplied to a thermal flying height control heater versus temperature during calibration.

FIGS. 8A and 8B are charts comparing helium concentration calculated by example techniques versus actual helium concentration for different hard disk heads.

FIG. 9A is a chart presenting an example estimated helium concentration calculated based on the thermal flying height.

FIG. 9B is a chart presenting an example estimated helium concentration calculated based on a change in the DC resistance exhibited by the ECS divided by the energy supplied to a thermal flying height control heater.

### DETAILED DESCRIPTION

The disclosure describes techniques for monitoring the composition of an atmosphere or a concentration of gas within an enclosure of a hard disk drive (HDD). In some examples, sealed storage devices may be filled with a gas, such as helium. Gas-filled HDDs may have advantages compared to unsealed storage devices, such as reduced power consumption, reduced mechanical vibration, and a higher storage density by allowing higher number of platters to be enclosed in a single device. Gas-filled HDDs may also be hermetic, reducing or substantially eliminating contamination from external dust or other sources that may degrade hard-drive performance or corrupt stored data. Further, using gases such as helium instead of air in the sealed enclosures may provide advantages, for example, by presenting tower density than air, which may lead to lower fluidic resistance during spinning of the magnetic media and reducing turbulence. However, a loss of integrity of the sealed enclosure may lead to leakage of gas from or introduction of air into the sealed enclosure, which may result in premature wear, performance degradation, data loss, or failure. Introduction of air may also result in catastrophic failure, for example, by a loss of flying-height control because of density and flow changes leading to contact between read-write heads and disks. Therefore, monitoring the atmosphere composition or gas concentration within a HDD enclosure in real-time may help in detecting gas leakage and preventing data loss, for example, by switching off the unit in response to detecting a predetermined amount of leakage.

In some examples, the techniques for monitoring atmosphere composition or gas concentration within a storage device enclosure may utilize existing components within a storage device. For example, a storage device read-write head or slider may include an embedded contact sensor (ECS) and a thermal flying height control (TFC) heater to control the flying height of the read-write head relative to the disk or platter surface. For example, the heater may heat the slider, so that thermal expansion of the slider or protrusion of slider elements such as a read element may be controlled, ultimately controlling the separation between surfaces of the slider and the disk or platter surface. The TFC heater may heat the slider or environment adjacent the read-write head to control the flying height, and a DC resistance (DCR) sensor in the ECS may exhibit a resistance indicative of a temperature adjacent the read-write head.

The disclosure describes using the ECS and the TFC heater to monitor the atmosphere composition or gas concentration based on the thermal conductivities of the atmosphere filling the enclosure and air. The atmosphere composition or gas concentration may be indicative of an integrity of the gas-filled storage device enclosure. For example, for a helium-filled HDD enclosure, the thermal conductivity of helium (0.144 Wm⁻¹K⁻¹ at 25° C.) is about six times greater than that of air (0.024 Wm⁻¹K⁻¹ at 25° C.). This difference in thermal conductivity may result in different heat dissipation depending on the concentration of helium relative to any air present in the enclosure. For example, when the TFC heater heats the read-write head, the difference in heat dissipation resulting from the different thermal conductivity of the surrounding gas may manifest in temperature differences adjacent the read-write head in a substantially pure helium atmosphere compared to a helium atmosphere contaminated with air or an air atmosphere. Thus, the concentration of helium or detect leakage of air into the helium-tined enclosure may be measured using difference in ECS temperature caused by the different thermal conductivities of helium and air. In some examples, the ECS and TFC heater may be used to monitor the gas composition or concentration when the read-write head is off-disk and on-ramp, for example, when the disk is stationary and when the ECS and TFC heater are not actively being used to control the flying height of the read-write head.

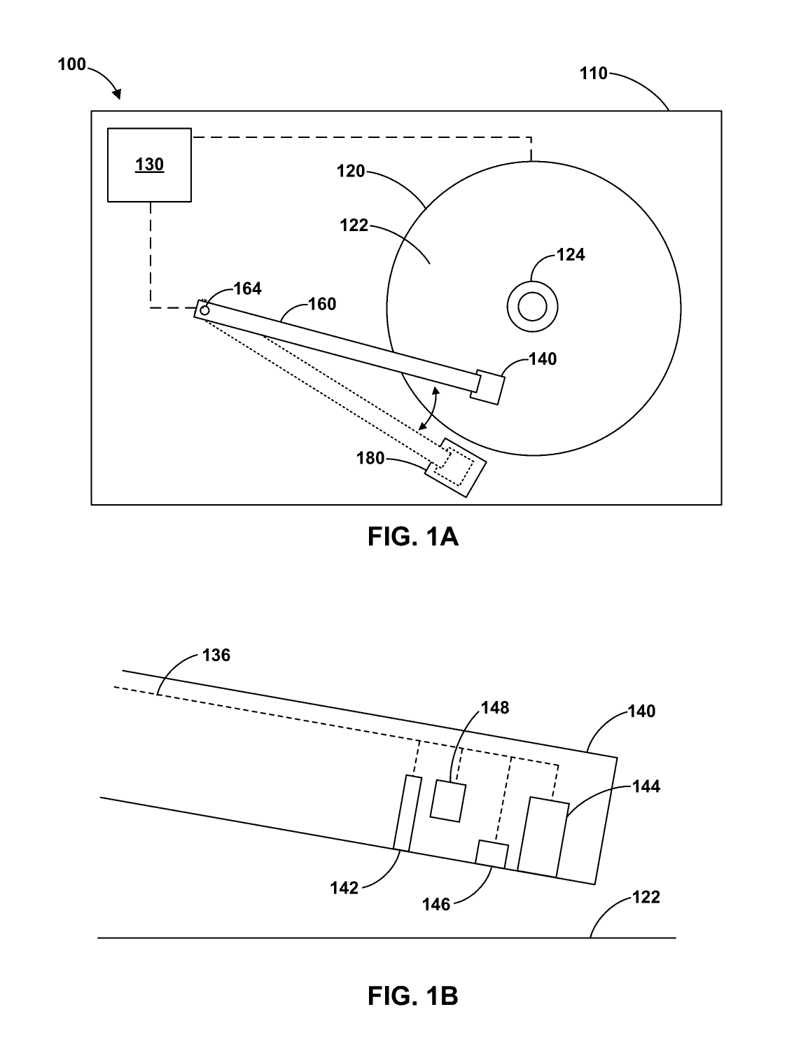

FIG. 1A is a conceptual and schematic block diagram illustrating an example storage device 100 including a storage disk 120 and a read-write head 140. Storage device 100 includes a seated enclosure 110 that encloses a storage medium, such as a storage disk 120 having a disk surface 122. Storage disk 120 may be mounted on a spindle 124, which may be connected to a spindle motor (not shown) for spinning storage disk 120. Sealed enclosure 110 also encloses a read-write head 140, an arm 160, a pivot 164, and a controller 130.

Sealed enclosure 110 may include a metal, polymer, or other suitable rigid housing that encloses storage disk 120. Sealed enclosure 110 may include a sealed atmosphere having a predetermined composition. In some examples, sealed enclosure 110 may enclose an atmosphere including a selected gas, which may be substantially free of air (e.g., nitrogen and oxygen). In some examples, at the time of manufacture, the atmosphere within sealed enclosure 110 may consist essentially of or consist of the selected gas. The selected gas may include, for example, helium.

Storage device 100 also includes read-write head 140, which may be disposed at an end of arm 160. When storage disk 120 spins, the spinning may create a cushion of gas on which read-write head 140 rides so that read-write head 140 flies above disk surface 122 without making contact with the magnetic medium of storage disk 120 in which information is recorded.

Arm 160 is mounted on a pivot 164, which defines an axis about which arm 160 pivots. In some examples, pivot 164 may include a voice-coil actuator or other type of rotational actuator to rotate arm 160 and read-write head 140 about pivot 164.

Storage device 100 also includes a controller 130, which may communicate with and control the spindle motor to control spinning of storage disk 120, read-write head 140, and a rotational actuator that controls movement of arm 160 about pivot 164. For example, controller 130 may control the spindle motor to control a spin speed of disk 120, including speeding up disk 120 to a predetermined speed, or braking disk 120 to a stop.

Controller 130 further may control a read operation or a write operation from the read-write head 140 onto the disk surface 122, e.g., through a combination of controlling positioning of read-write head 140 relative to storage disk 120 and controlling operation of read-write head 140 to write data or read data. Controller 130 may control an orientation or position of arm 160 and read-write head 140 relative to the disk surface 122 with respect to spindle 124, so that read-write head 140 may access different tracks of data stored by storage disk 120. Controller 130 may control the rotational actuator to cause arm 160 to be moved towards or away from spindle 124 to position the read-write head 140 at a location adjacent disk surface 122 at a predetermined radial distance from spindle 124. Similarly, controller 130 may control the spindle motor to spin storage disk 120 to position read-write head 140 over a selected circumferential position of storage disk 120. In this way, controller 130 may control positioning of read-write head 140 over selected positions of storage disk 120. Controller 130 then may cause data to be written to or read from storage disk 120 using read-write head 140. In some examples, when read-write head 140 is not being used to read data from or write data to storage disk 120, controller 130 may cause the rotational actuator to move arm 160 to position read-write head 140 off-disk in a resting position adjacent to storage disk 120, such as on a ramp 180 in storage device 100. This may reduce a likelihood of read-write head 140 contacting storage disk 120 in the event of a physical shock to storage device 100.

In some examples, controller 130 may be enclosed within the enclosure 110, as shown in FIG. 1A. However, controller 130 may be disposed outside enclosure 110, and may be electronically connected, for example, by wires or wirelessly, to at least one of the rotational actuator controlling motion of arm 160, the spindle motor controlling rotational motion of storage disk 120, or read-write head 140.

FIG. 1B is a conceptual and schematic block diagram illustrating a cross-sectional view of read-write head 140 of FIG. 1A. While 1B shows read-write head 140 adjacent disk surface 122, in other examples, read-write head 140 may be disposed adjacent to or on ramp 180, as described above. As shown in FIG. 1B, read-write head 140 may include a read sensor 142 and a write device 144. Read-write head 140 also includes an embedded contact sensor (ECS) 146. ECS 146 may include a DC resistance (DCR) sensor that exhibits a resistance indicative of a temperature adjacent ECS 146, and may output a signal indicative of the sensed temperature. For example, the DCR sensor of ECS 146 may exhibit a decreased resistance in response to a higher temperature, and may exhibit an increased resistance in response to a lower temperature. In other examples, the DCR sensor of ECS 146 may exhibit an increased resistance in response to a lower temperature, and may exhibit a decreased resistance in response to a higher temperature. In some examples, the signal output by ECS 146 may be indicative of contact between read-write head 1.40 and disk surface 122 that may result in elevated temperatures due to frictional heat between read-write head 140 and disk surface 122.

Read-write head 140 further includes a thermal flying-height control (TFC) heater 148. The spacing between read-write head 140 and disk surface 122 may affect the performance of the storage device 100, for example, by affecting error rates in data written to or read from storage disk 120. Read sensor 142, write device 144, or both may protrude from read-write head 140 toward disk surface 122, and this protrusion may change with, for example, temperature, affecting the spacing between read sensor 142 and disk surface 122, between write device 114 and disk surface 122, or both.

Thermal flying-height control may be used to control the spacing between disk surface 122 and read sensor 142 or write device 144. The spacing may be affected by factors including the write current supplied to write device 144, which may lead to heating causing write device 144 to expand in size and approach disk surface 122. Thus, the effective spacing between read sensor 142 and disk surface 122, and between write device 114 and disk surface 122 may change as storage device 100 is operated. Should the spacing become too small, one or more components of read-write head 140 may contact disk surface 122, which may lead to damage of read-write head 140, storage device 120, or both, and loss of data or failure of storage device 100. TFC heater 148 may be used to compensate for thermal effects of reading and writing and adjust spacing between read-write head 140 and disk surface 122 to maintain the selected flying-height and avoid unwanted contact between components of read-write head 140 and disk surface 122. For example, a controlled electrical current may be constantly applied to TFC heater 148 in operation to maintain a predetermined temperature and flying-height, and the applied current may be reduced to compensate for additional heat generated by a write current applied to write device 144, additional heat generated by a read current applied to read sensor 142, or both.

In some examples, controller 130 may communicate with and control each of read sensor 142, write device 144, ECS 146, and TFC heater 148 using signals carried by a bus 136 as shown in FIG. 1B. For example, controller 130 may cause a current to be applied to TFC heater 148 to generate heat. Controller 130 may receive a signal from ECS 146 indicative of the DC resistance exhibited in response to the temperature sensed by the DCR sensor.

In accordance with one or more techniques of this disclosure, in addition to being used to control flying-height of read-write head 140, controller 130 may implement techniques to determine an atmosphere composition or gas concentration within sealed enclosure 110 using TFC heater 140 and ECS 146. For example, sealed enclosure 110 may enclose an atmosphere including a known initial concentration of a predetermined gas, such as helium. In examples, controller 130 may determine the present concentration of gas using EQUATION 1:

X=GASᵢₙᵢₜᵢₐₗ+(Slope_{measured}−C(T−T₀)−S₀)/A (EQUATION 1)

In EQUATION 1, X is the present concentration of gas in sealed enclosure 110 in percent volume to be determined. When controller 130 causes a current to be applied to TFC heater 148, TFC heater 148 generates heat that is dissipated in the vicinity of TFC heater 148. The dissipated heat may affect the temperature in a region adjacent read-write head 140, and the temperature may depend on the gas concentration enclosed in sealed enclosure 110, because different gases have different thermal conductivities and heat capacities, and thus change temperature more or less when presented with the same heat load.

The DCR sensor in ECS 146 may exhibit a change in the DC resistance in response to the changed temperature. The Slope_{measured} in EQUATION 1 is a present observed change in the DC resistance divided by the total energy supplied by the predetermined current applied to TFC heater 148 at a present temperature T. S₀ is a change in the DC resistance divided by the total energy supplied by a predetermined current applied to the TFC heater 148 at a reference concentration of gas GASᵢₙᵢₜᵢₐₗ and at a reference temperature T₀.

C is a first predetermined calibration constant, and A is a second predetermined calibration constant. The calibration constant A may be determined by evaluating the slope of a hypothetical tine that passes through calibration points representing respective changes in DC resistance divided by respective total energy supplied to TFC heater 148 as a function of known gas concentration at the reference temperature T₀, as explained with respect to FIG. 6A below. For example, calibration points may be measured at gas concentrations of 50%, 70%, 80%, 90%, and 98% by volume, and the slope of a line that passes through the calibration points may be evaluated. The calibration constant C may be determined by determining the slope of a hypothetical line that passes through calibration points representing respective changes in DC resistance divided by respective total energy supplied to TFC heater 148 as a function of temperature, as explained with respect to FIG. 6B below. For example, calibration points may be measured at temperatures of 5° C., 25° C., and 60° C., and the slope of a line that passes through the calibration points may be determined.

While examples have been described above with respect to disk 120 and read-write head 140, in some examples, sealed enclosure 110 may enclose more than one disk and read-write head, and the DC resistance may be monitored at one or more read-write heads to establish the atmosphere composition or gas concentration. For example, the gas concentrations estimated using the ECS at different heads may be averaged, or the maximum or minimum of estimated gas concentrations from different ECS units may be used.

Thus, controller 130 may implement techniques to determine an atmosphere composition or gas concentration within seated enclosure 110 using TFC heater 148 and ECS 146. In some examples, if controller 130 receives a signal from ECS 146 indicative of a temperature change, controller 130 may determine the present concentration of gas using EQUATION 1. In some examples, controller 130 may determine the concentration of gas at predetermined intervals of time In some examples, controller 130 may control TFC heater 148 to change the amount of heat supplied in response to the detected gas concentration. For example, if controller 130 detects a relatively small change in gas concentration, controller 130 may adjust the algorithm for controlling the flying height or the head-to-disk spacing, for example, by changing the power supplied to the TFC heater 148. In some examples, if controller 130 detects a relatively large change in atmosphere composition or gas concentration, for example, above the threshold, controller 130 may move read-write head 140 to ramp 180 to prevent damage to disk 120, or may communicate an error message to a host device to alert the host device of a problem and potential failure of storage device 100. In this way, errors or malfunctions due to changes in atmosphere within the enclosure may be reduce or substantially eliminated.

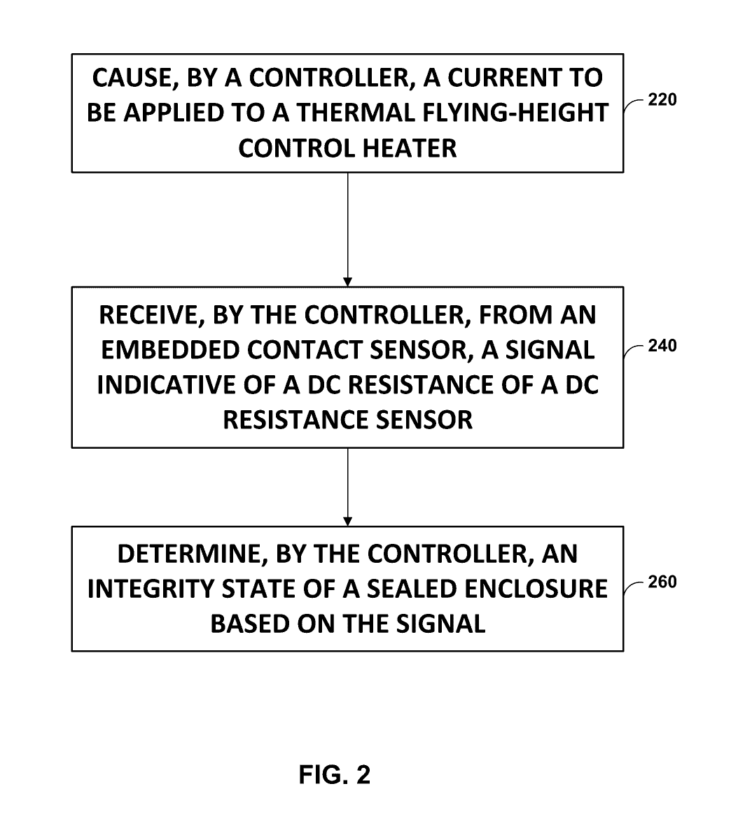

FIG. 2 is a flow diagram illustrating an example technique for detecting a composition of an atmosphere or a concentration of a gas within a sealed enclosure of a storage device. While example techniques below are described with reference to storage device 100 of FIGS. 1A and 1B, the techniques may be implemented in other example articles or systems. In some examples, the example technique may include causing, by controller 130, a current to be applied to TFC heater 148 (220). The current may cause TFC heater 148 to heat read-write head 140, which heat may pass to the surrounding gas or atmosphere, for example, by conduction. The heat passing to the surrounding atmosphere may cause the atmosphere to assume a temperature that depends on the thermal conductivity and specific heat capacity of the atmosphere. For example, if the atmosphere has a relatively low thermal conductivity, the temperature may rise to a relatively greater extent and if the atmosphere has a relatively high thermal conductivity, the temperature may rise to a relatively lower extent. If the atmosphere has a relatively low specific heat capacity, the temperature may rise to a relatively greater extent, and if the atmosphere has a relatively high heat capacity, the temperature may rise to a relatively lower extent. In some examples, the DCR sensor of ECS 146 may exhibit a changed resistance in response to the sensed temperature, and thus ECS 146 may generate a signal indicative of the temperature.

The technique of FIG. 2 also may include, receiving, by controller 130, from ECS 146, a signal indicative of a DC resistance of the DCR sensor (240). The DC resistance may be indicative of a temperature adjacent the DCR sensor. The example technique may include determining, by controller 130, a composition of the atmosphere or a concentration of the gas within sealed enclosure 110 based on the signal (26). For example, sealed enclosure 110 may enclose an initial concentration of the gas, and controller 130 may determine a present concentration of the gas based on the signal. The present concentration of the gas may be indicative of an integrity state of sealed enclosure 110, as changes in the present concentration of the gas may be indicative of a breach or leak in sealed enclosure 110. As described in examples above with reference to FIG. 1, controller 130 may determine the present concentration of the gas using EQUATION 1, for example, to ultimately determine the atmosphere composition or gas concentration.

In some examples, the example technique may further include, generating by controller 130, an output indicative of the atmosphere composition or gas concentration, or of integrity state determined based on the atmosphere composition or gas concentration. For example, controller 130 may generate an output indicative of the current gas composition or concentration within sealed enclosure 110. In some examples, controller 130 may output an electronic signal indicative of the integrity state. In some examples, controller 130 may send the electronic signal to a host device. For example, controller 130 may send an output to the host device to alert the host device of a change in gas concentration beyond a predetermined threshold that may be indicative of leakage in the sealed enclosure or that may be indicative of imminent failure of storage device 100.

In some examples, storage disk 120 includes a magnetic data storage disk, and the rest position includes resting read-write head 140 on ramp 180. Controller 130 may send signals that cause read-write head 140 move to a rest position and determine the integrity state in the rest position. For example, controller 130 may determine the atmosphere composition or gas concentration white read-write head 140 is in the rest position while storage disk 120 is spinning In some examples, controller 130 may determine the atmosphere composition or gas concentration while read-write head 140 is in the rest position while storage disk 120 is stationary. Thus, in some examples, the example technique may further include controlling, by controller 130, read-write head 140 to move to a rest position, and determining, by controller 130, the atmosphere composition or gas concentration while read-write head 140 is in the rest position.

Thus, controller 130 may implement the example technique of FIG. 2 to determine gas concentration or atmosphere composition within sealed enclosure 110 of storage device 100 of FIG. 1. Further, controller 130 may implement the example technique of FIG. 2 to detect leakage or breach of sealed enclosure 110 by determining the gas concentration or atmosphere composition.

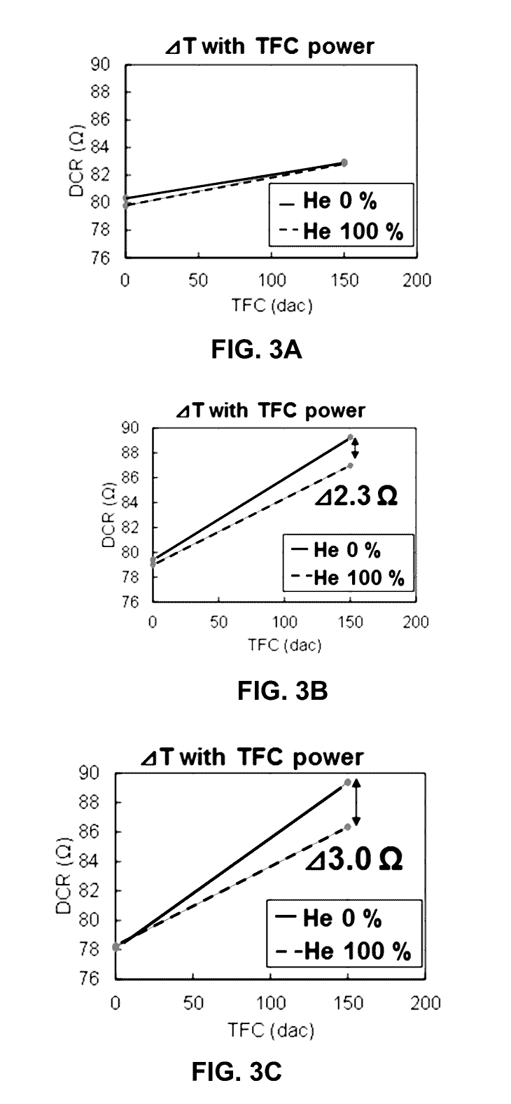

FIGS. 3A-3C are charts presenting an example DC resistance versus energy supplied to a thermal flying height control heater as a function of helium concentration, and at different positions of the read-write head with respect to the disk and the ramp. The DC resistance (DCR) was measured when no power was supplied to the TFC heater (TFC=0) and when power of 150 dac (where 1 dac=0.165 MW) was supplied to the TFC heater. The ECS bias was 1.4 mA. FIG. 3A represents a DC resistance measured when the read-write head was over the disk (adjacent the disk surface). The DC resistance did not change with helium concentration when the TFC heater was at a power of 150 dac.

FIG, 3B represents a DC resistance measured when the read-write head was on the ramp (rest position, off-disk) with the disk rotating. The DC resistance increased by 2.3Ω when helium concentration reduced from 100% to 0%, when the TFC heater was at a power of 150 dac. FIG. 3C represents a DC resistance measured when the read-write head was on the ramp (rest position, off-disk) with a stationary disk (no rotation). The DC resistance increased by 3.0Ω when helium concentration reduced from 100% to 0%, when the TFC heater was at a power of 150 dac. Thus, in comparison between the examples illustrated in FIGS. 3A-3C, the change in DC resistance (ΔDCR) was most indicative of a change in helium concentration when the head was on the ramp and the disk was stationary.

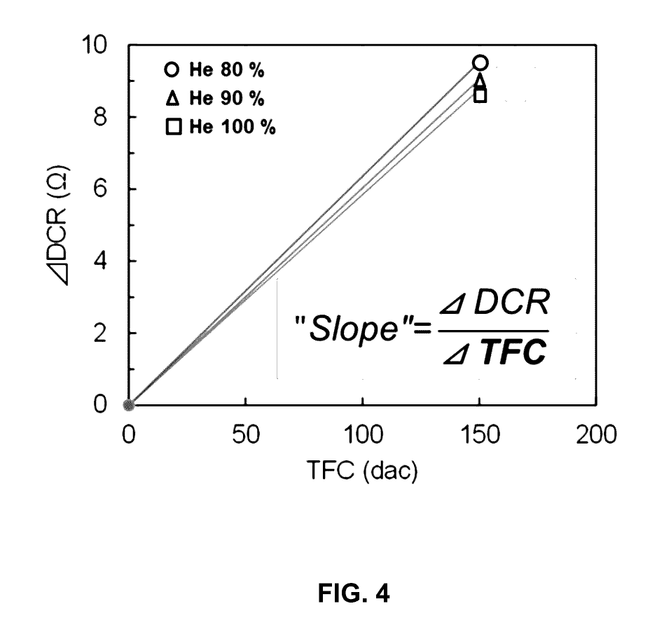

FIG. 4 is a chart presenting an example change in DC resistance versus energy supplied to a thermal flying height control heater as a function of TFC power for different helium concentrations. The change in the DC resistance (ΔDCR) of an ECS of a read-write head was measured at different concentrations of helium within an enclosure, with unpowered and powered TFC heater (TFC, measured in dac). As shown in FIG. 4, there was no change in the DC resistance when comparing different helium concentrations when the TFC heater was unpowered. The ΔDCR increased with a reduction in the helium concentration. A “slope” was established for each respective helium concentration by dividing the change in ΔDCR by the change in TFC.

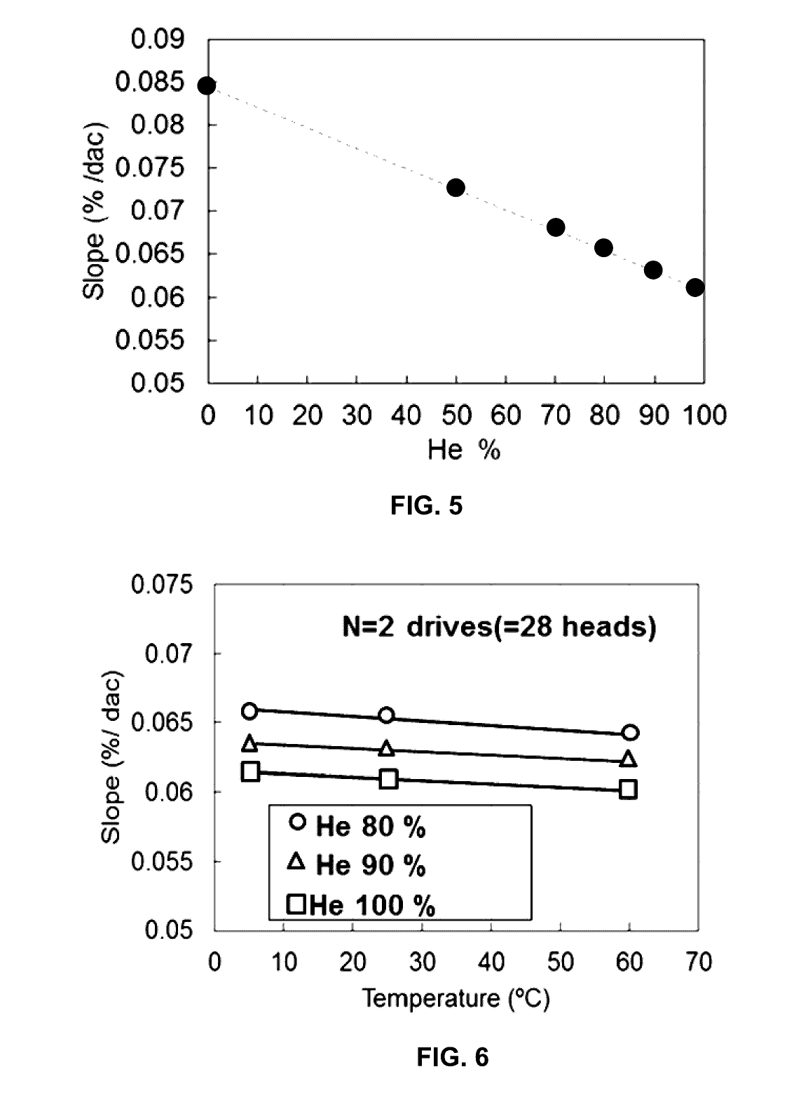

FIG. 5 is a chart presenting an example percent change in ΔDC resistance divided by change in energy supplied to a thermal flying height control heater for different helium concentrations. The “slope” (y-axis value in FIG. 5) was calculated from the ΔDCR measurement (similar to FIG. 4), and increased linearly with a reduction in helium concentration. The temperature was 2.5° C., and data was averaged from 14 read-write heads.

FIG. 6 is a chart presenting an example change in ΔDC resistance divided by change in energy supplied to a thermal flying height control heater versus temperature for different helium concentrations. The “slope” (y-axis value in FIG. 6) was calculated from the ΔDCR measurement (similar to FIG. 4), and decreased linearly with increasing temperature, for each respective helium concentration of 100%, 90% and 80% by volume.

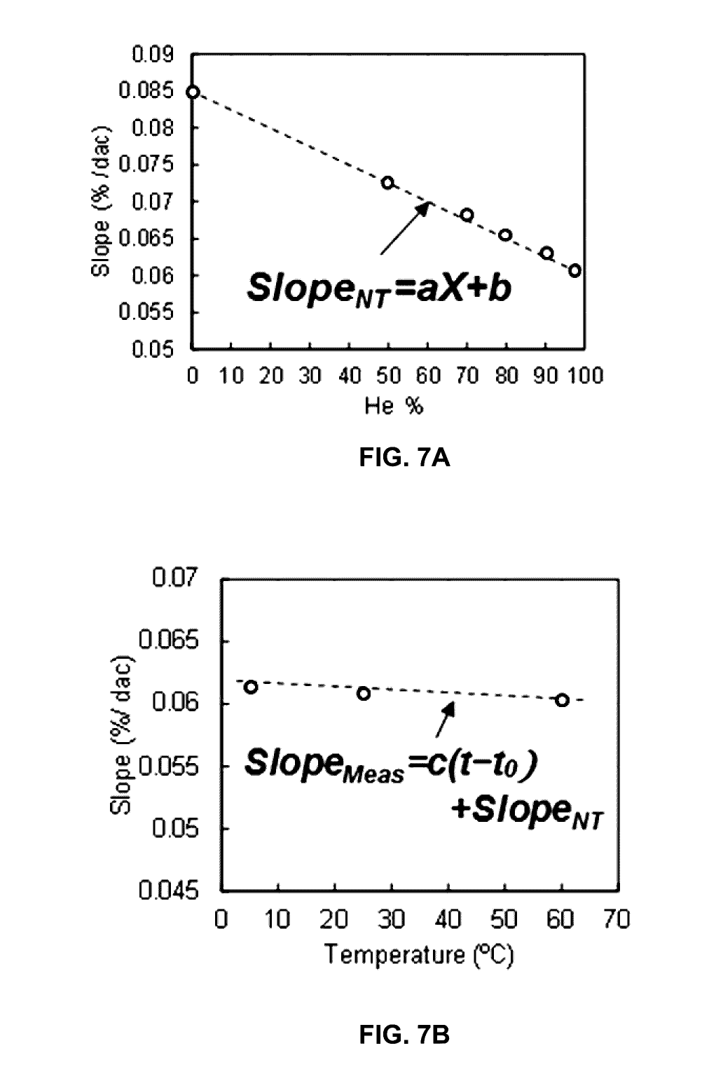

FIG. 7A is a chart presenting an example change in ΔDC resistance divided by change in energy supplied to a thermal flying height control heater versus helium concentration during calibration. The “slope” (y-axis value in FIG. 7A) was measured at different helium concentrations and a reference or constant temperature of 25° C., and a tine was plotted through the different values of the “slope”. This line (expressed in terms of the linear formula Slope_{NT}=aX+B, where Slope_{NT} is the “slope” at NT or Normal Temperature, T=25° C.) was used to find the “slope” at different helium concentrations, and the geometric slope of this line (“a”) corresponds to the calibration constant “A” in EQUATION 1.

FIG. 7B is a chart presenting an example change in ΔDC resistance divided by change in energy supplied to a thermal flying height control heater versus temperature during calibration. The “slope” (y-axis value, in FIG, 7B) was measured at different temperatures for a reference or constant helium concentration, and a line was plotted through the different values of the “slope”. This line (expressed in terms of the linear formula Slopeₘₑₐₛ=c(t−t₀)+Slope_{NT}, where Slopeₘₑₐₛ is the “slope” measured at a given temperature t, and t₀ is the reference temperature (here, NT or 25° C.) can be used to relate the “slope” measured at a given temperature to the helium concentration corresponding to that slope. The geometric slope of this line (“c”) corresponds to the calibration constant “C” in EQUATION 1.

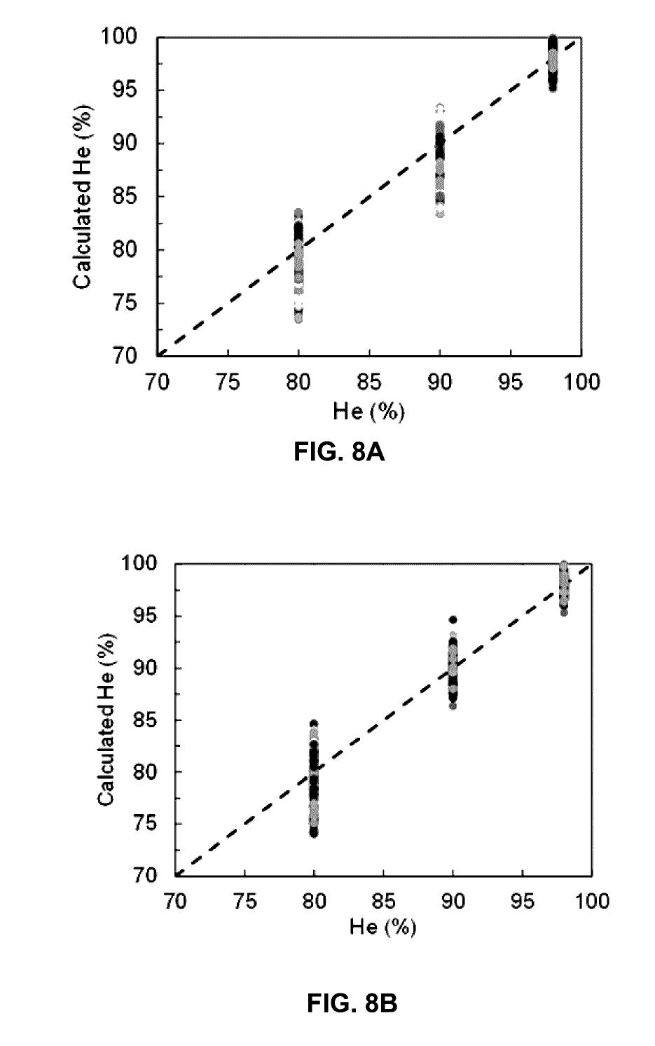

FIGS. 8A and 8B are charts comparing helium concentration calculated by example techniques versus actual helium concentration for different hard disk heads. In FIG. 8A, the helium concentration was calculated for 98 heads of a first type using EQUATION 1, and plotted against the actual known helium concentrations to find the error in the calculated helium % relative to the actual helium % in %volume. In FIG. 8B, the helium concentration was calculated for 112 heads of a second type using EQUATION 1, and plotted against the actual known helium concentrations to find the error in the calculated helium % relative to the actual helium % in %volume. As shown in FIGS. 8A and 8B, the error (calculated value actual value) was in a relatively narrow range, with a maximum variance of 8% for both types of heads.

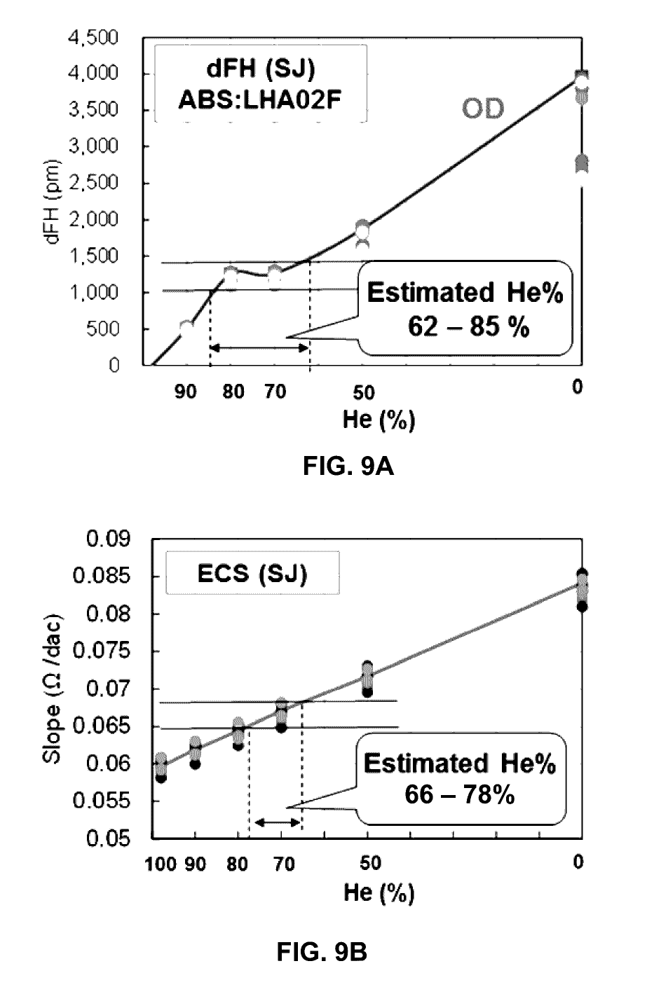

FIG. 9A is a chart presenting an exarriple estimated helium concentration calculated based on the thermal flying height. The change in the flying height (dFH, measured in picometers) was plotted as function of the helium concentration, for heads of the second type. As shown in FIG. 9A, the relation between helium concentration and the change in the flying height is non-linear, making it difficult to establish with accuracy the helium concentration that corresponds to a particular change in flying height. For example, a dFH between 1000 and 1500 pm may correspond to an estimated helium concentration that may range anywhere between 62 to 85%, a relatively wide variance, with a maximum variance of up to 15%.

FIG. 9B is a chart presenting an example estimated helium concentration calculated based on a change in the DC resistance exhibited by the ECS divided by the energy supplied to a thermal flying height control heater. In contrast with FIG. 9A, the “slope” (determined for example, according to the examples of FIGS. 4 to 6) had a linear relation with helium concentration, whereby the estimated helium concentration varied in a relatively narrower band, from 74 to 88%, allowing greater accuracy in determining helium concentration (±8%) compared to the technique of FIG. 9A.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term. “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described. These and other examples are within the scope of the following claims.

What is claimed is:

1. A storage device comprising:
a sealed enclosure enclosing a storage medium and a read-write head in an atmosphere comprising a gas, the read-write head comprising a thermal flying-height controller (TFC) heater and an embedded contact sensor (ECS) comprising a DC resistance (DCR) sensor, wherein a DC resistance exhibited by the DCR sensor is indicative of a temperature sensed adjacent the DCR sensor; and
a controller, wherein the controller is configured to:
cause a current to be applied to the TFC heater to generate heat;
receive a signal from the ECS indicative of the temperature sensed by the DCR sensor; and
determine a composition of the atmosphere or a concentration of the gas based on the signal.

2. The storage device of claim 1, wherein the sealed enclosure encloses an initial concentration of the gas, and the composition of the atmosphere or the concentration of the gas comprises a present concentration of the gas in the sealed enclosure.

3. The storage device of claim 2, wherein the controller is configured to determine present concentration of the gas using the formula:
X−GASᵢₙᵢₜᵢₐₗ+(Slope_{measured}−C(T−T₀)−S₀)/A
wherein X is the present concentration of the gas in % volume, GASᵢₙᵢₜᵢₐₗ is the initial concentration of the gas, T is a present temperature sensed by the DCR sensor, Slope_{measured} is a present observed change in the DC resistance divided by a total energy supplied by the current applied to the TFC heater at the present temperature T, S₀ is a known change in the DC resistance divided by the total energy supplied by the current applied to the TFC heater at the reference concentration of the gas GASᵢₙᵢₜᵢₐₗ and a reference temperature T₀, C is a first predetermined calibration constant, and A is a second predetermined calibration constant.

4. The storage device of claim 1, wherein the controller is further configured to generate an output indicative of the composition of the atmosphere or the concentration of the gas.

5. The storage device of claim 1, wherein the controller is configured to move the read-write head to a rest position and determine the composition of the atmosphere or the concentration of the gas in the rest position.

6. The storage device of claim 5, wherein the storage device comprises a ramp, the storage medium comprises a magnetic data storage disk, and the rest position comprises resting the read-write head on the ramp.

7. The storage device of claim 6, wherein the controller is configured to determine the composition of the atmosphere or the concentration of the gas in the rest position while the magnetic data storage disk is stationary.

8. The storage device of claim 1, wherein the gas comprises helium.

9. A method comprising:
causing, by a controller, a current to be applied to a thermal flying-height control (TFC) heater, wherein a sealed enclosure encloses a storage medium and a read-write head in an atmosphere comprising a gas, the read-write head comprising the TFC heater and an embedded contact sensor (ECS) comprising a DC resistance (DCR) sensor,
receiving, by the controller, from the ECS, a signal indicative of a DC resistance of the DCR sensor, wherein the DC resistance is indicative of a temperature adjacent the DCR sensor; and
determining, by the controller, a composition of the atmosphere or a concentration of the gas based on the signal.

10. The method of claim 9, wherein the sealed enclosure encloses an initial concentration of the gas, and the composition of the atmosphere or the concentration of the gas comprises a present concentration of the gas.

11. The method of claim 10, wherein the determining the composition of the atmosphere or the concentration of the gas comprises, by the controller, determining the present concentration of the gas using the formula:
X=GASᵢₙᵢₜᵢₐₗ+(Slope_{measured}−C(T−T₀)−S₀)/A
wherein X is the present concentration of the gas in % volume, Slope_{measured} is a present observed change in the DC resistance divided by the total energy supplied by the current applied to the TFC heater at a present temperature T, S₀ is a known change in the DC resistance divided by the total energy supplied by the current applied to the TFC heater at a reference concentration of the gas GASᵢₙᵢₜᵢₐₗ and at a reference temperature T₀, C is a first predetermined calibration constant, and A is a second predetermined calibration constant.

12. The method of claim 9, thither comprising, by the controller, generating an output indicative of the composition of the atmosphere or the concentration of the gas.

13. The storage device of claim 9, further comprising, by the controller, moving the read-write head to a rest position, and determining, by the controller, the composition of the atmosphere or the concentration of the gas in the rest position.

14. The method of claim 13, wherein the storage device comprises a ramp, the storage medium comprises a memory disk, and the rest position comprises resting the read-write head on the ramp.

15. The method of claim 14, wherein the memory disk is stationary.

16. The method of claim 9, wherein the gas comprises helium.

17. A system comprising a storage device comprising:
a sealed enclosure enclosing a storage medium and a read-write head in an atmosphere comprising a gas, the read-write head comprising a means for electrically heating a region adjacent the read-write head and a means for exhibiting a DC resistance (DCR) indicative of a temperature sensed adjacent the read-write head; and a controller, the controller comprising:
a means for applying a current to be applied to the electrical means;
a means for receiving a signal from the means for exhibiting the DC resistance; and
a means for determining a composition of the atmosphere or a concentration of the gas based on the signal,

18. The system of claim 17, wherein the means for determining he composition of the atmosphere or the concentration of the gas comprises means for evaluating the formula:
X=GASᵢₙᵢₜᵢₐₗ+(Slope_{measured}−C(T−T₀)−S₀)/A
wherein X is the present concentration of the gas in % volume, Slope_{measured} is a present observed change in the DC resistance divided by the total energy supplied by the current applied to the TFC heater at a present temperature T, S₀ is a known change in the DC resistance divided by the total energy supplied by the current applied to the TFC heater at a reference concentration of the gas GASᵢₙᵢₜᵢₐₗ and at a reference temperature T₀, C is a first predetermined calibration constant, and A is a second predetermined calibration constant.

19. A computer readable storage medium comprising instructions that, when executed, cause at least one processor to:
cause a current to be applied to a thermal flying-height control (TFC) heater, wherein a sealed enclosure encloses a storage medium and a read-write head in an atmosphere comprising a gas, the read-write head comprising the TFC heater and an embedded contact sensor (ECS) comprising a DC resistance (DCR) sensor,
receive, from the ECS, a signal indicative of a DC resistance of the DCR sensor, wherein the DC resistance is indicative of a temperature adjacent the DCR sensor; and
determine a composition of the atmosphere or a concentration of the gas based on the signal.

20. The computer readable storage medium of claim 19, further comprising instructions that, when executed, cause the at least one processor to determine the composition of the atmosphere or the concentration of the gas by determining the present concentration of the gas using the formula:
X=GASᵢₙᵢₜᵢₐₗ+(Slopeₘₑₐₛᵤᵣₑₘₑₙₜ−C(T−T₀)−S₀)/A
wherein X is the present concentration of the gas in % volume, Slope_{measured} is a present observed change in the DC resistance divided by the total energy supplied by the current applied to the TFC heater at a present temperature T, S₀ is a known change in the DC resistance divided by the total energy supplied by the current applied to the TFC heater at a reference concentration of the gas GASᵢₙᵢₜᵢₐₗ and at a reference temperature T₀, C is a first predetermined calibration constant, and A is a second predetermined calibration constant.
